(19) United States
(12) Patent Application Publication
(10) Pub. No.: US 2002/0183291 A1
(43) Pub. Date: Dec. 5, 2002

(54) Product for treating gynecomastia

(76) Inventors: Giorgio Massimini, Abbiategrasso (IT); Gabriella Piscitelli, Milano (IT)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 10/148,902

(22) PCT Filed: Dec. 8, 2000
(86) PCT No.: PCT/EP00/12533

(30) Foreign Application Priority Data
Dec. 30, 1999 (GB) 9930839.7

(51) Int. Cl. A61K 31/56

(52) U.S. Cl. 514/170; 514/179

(57) ABSTRACT

The invention provides a method of treating gynecomastia in a patient being treated with an antiandrogen, which method comprises administering to a patient in need thereof a therapeutically effective amount of an aromatase inhibitor selected from exemestane, formestane, anastrozole, letrozole and fadrozole, or a pharmaceutical composition comprising a said aromatase inhibitor. The use of a said aromatase inhibitor in the preparation of a pharmaceutical composition for treating gynecomastia induced by antiandrogen therapy and a kit containing the said aromatase inhibitor and an antiandrogen are also provided.

### BACKGROUND

This invention relates to a method of treating gynecomastia in patients being treated with an antiandrogen, who are in need of such treatment. This invention more particularly relates to a method of treating gynecomastia in prostatic cancer and in benign prostatic hypertrophy patients being treated with an antiandrogen, who are in need of such treating, comprising administering to such patients in association with therapeutically effective amounts of the antiandrogen, an aromatase inhibitor selected from exemestane, formestane, anastrozole, letrozole and fadrozole, or pharmaceutical compositions thereof.

Prostatic cancer has been treated with antiandrogens. Benign prostatic hypertrophy, a particularly common problem in older men, has been treated with antiandrogens and by use of estrogenic substances. The use of estrogenic substances has undesirable, life-threatening side effects due to the inherent properties of the estrogenic substances.

The use of antiandrogens, e.g. bicalutamide, flutamide, nilutamide and cyproterone acetate, while devoid of life-threatening side effects associated with treatment with estrogenic substances, produces gynecomastia in up to 40% of non-castrated males studied, including normal subjects and advanced prostate cancer patients. Gynecomastia interferes with continued patient compliance with the antiandrogen therapy.

Accordingly, there is a need in therapy for a method of treating gynecomastia in patients being treated with an antiandrogen, e.g. bicalutamide, flutamide, nilutamide and cyproterone acetate, who are in need of such treatment.

### SUMMARY OF THE INVENTION

The invention provides a method of treating gynecomastia in patients being treated with an antiandrogen in need of such treating, which comprises administering to such patients a therapeutically effective amount of an aromatase inhibitor selected from exemestane, formestane, anastrozole, letrozole and fadrozole, or a pharmaceutical composition thereof.

### DETAILED DESCRIPTION OF THE INVENTION

In one preferred aspect, the present invention provides an effective method of treating gynecomastia in patients, being treated with an antiandrogen to treat androgen-dependent or androgen-caused disease states, which patients are in need of such treating, by administering therapeutically effective amounts of the antiandrogen in association with an aromatase inhibitor selected from exemestane, anastrozole, letrozole and fadrozole, or pharmaceutical compositions thereof.

Examples of such androgen-dependent or androgen-caused disease states are prostatic cancer and benign prostate hypertrophy.

Accordingly, in another aspect, the present invention provides a method of treating gynecomastia in prostatic cancer or benign prostatic hypertrophy patients in need of such treating, which comprises administering to such patients in association with a therapeutically effective amount of an antiandrogen a therapeutically effective amount of an aromatase inhibitor selected form exemestane, formestane, anastrozole, letrozole and fadrozole or pharmaceutical compositions thereof.

Preferred aromatase inhibitor is exemestane.

In a further aspect, the present invention provides the use of an aromatase inhibitor selected form exemestane, formestane, anastrozole, letrozole and fadrozole in the preparation of a medicament for use in treating gynecomastia in a patient undergoing a simultaneous, separate or sequential treatment with an antiandrogen.

In a further aspect, the present invention provides a kit comprising, in suitable container means, a pharmaceutical composition containing an antiandrogen, for instance as exemplified above, as the active agent, and a pharmaceutical composition containing an aromatase inhibitor selected form exemestane, formestane, anastrozole, letrozole and fadrozole, as the active agent, wherein the aromatase inhibitor is for use in treating gynecomastia in patients, being treated with the antiandrogen.

The antiandrogen and the aromatase inhibitor may be present within a single or distinct container means.

Preferably such kit contains antiandrogen bicalutamide and aromatase inhibitor exemestane.

According to the method of treatment of the present invention, the active compounds may be administered either together, separately, simultaneously or sequentially, in any order, as discussed hereinafter. Preferably the active compounds are administered substantially simultaneously.

By the term <<(patients in need of such treating>> is meant (<<male patients with functioning gonads who are being treated with antiandrogen and who exhibit or are likely to exhibit symptoms of gynecomastia>>. Male patients with functioning gonads have not been surgically castrated or chemically castrated by chronic administration of a LH-RH agonist or antagonist.

Preferred examples of antiandrogens according to the invention are: bicalutamide, cyproterone acetate, flutamide and nilutamide, in particular bicalutamide.

Androgen antagonist bicalutamide is known e.g. from EP-100172.

Cyproterone acetate and flutamide are known e.g. from Cancer Treat. Res., 94, 231-254, 1998. Nilutamide is known e.g. from The Merck Index, 12^{th} edition, see 6636.

By the term <<aromatase inhibitor>> is meant a substance that inhibits the aromatic conversion of tesosterone into estradiol. Aromatase inhibitors according to the present invention are fadrozole, letrozole, anastrozole, formestane, and exemestane. Exemestane, letrozole and anastrozole, in particular exemestane, being preferred examples of aromatase inhibitors according to the invention.

Letrozole, fadrozole, anastrozole formestane and exemestane are well known products, see e.g. Cancer. Treat. Res., 94, 231-254,1998.

In this invention, the antiandrogen and the aromatase inhibitor are administered as pharmaceutical compositions via parenteral or oral means. Preferably the antiandrogen and the aromatase inhibitor are administered orally.

The amount of each component administered is determined by the attending clinicians taking into consideration the etiology and severity of the disease, the patient's condition and age, the potency of each component and other factors.

For instance, the antiandrogen bicalutamide compositions are generally administered in a dosage range of about 10 to 200 mg per day, preferably from about 50 to about 150 mg per day.

The aromatase inhibitor compositions are generally administered in a dosage range of about 0.5 to about 500 mg/daily.

The letrozole compositions are generally administered orally in a dosage from about 0.5 to about 10 mg per day, preferably from about 0.5 to about 2.5 mg per day.

The anastrozole compositions are generally administered orally in a dosage from about 0.5 to about 10 mg per day, preferably from about 1 to about 2 mg per day. The exemestane compositions are generally administered orally in a dose from about 5 to about 200 mg per day, preferably from about 10 to about 25 mg per day, or parenterally from about 50 to about 500 mg, in particular from about 100 to about 250 mg.

The formestane compositions are generally administered parenterally from about 50 to about 500 mg, in particular from about 100 to about 250 mg.

In a preferred aspect of this invention, the antiandrogen bicalutamide is administered orally in a daily dose of about 50 mg and the aromatase inhibitor is exemestane, which is administered orally in a daily dose of about 25 mg.

The antiandrogen e.g. bicalutamide and the aromatase inhibitor e.g. exemestane may be compounded into a dosage form suitable for oral or parenteral administration or, provided in the form of a kit as described above.

A tablet or capsule or caplets are particularly convenient forms for oral administration. Such compositions useful in the present invention are typically formulated with conventional pharmaceutical excipients, e.g., spray dried lactose and magnesium stearate into tablets or capsules for oral administration. One or more of the active substances can be worked into tablets or dragee cores by being mixed with solid, pulverulent carrier substances, such as sodium citrate, calcium carbonate or dicalcium phosphate, and binders such as polyvinyl pyrrolidone, gelatin or cellulose derivatives, possibly by adding also lubricants such as magnesium stearate, sodium lauryl sulfate, <<Carbowax>>) or polyethylene glycols. Of course, taste improving substances can be added in the case of oral administration forms.

As further forms of administration, one can use plug capsules, e.g. hard gelatin, as well as closed soft gelatin capsules comprising a softner or plasticizer, e.g. glycerine. The plug capsules contain the active substance preferably in the form of a granulate, e.g., in mixtures with fillers, such as lactose, saccharose, mannitol, starches such as potato starch or amylopectin, cellulose derivatives or highly-dispersed silicic acids. In soft-gelatin capsules, the active substance is preferably dissolved or suspended in suitable liquids, such as vegetable oils or liquid polyethylene glycols.

In place of oral administration, the active compounds may be administered parenterally. In such case, one can use a dispersion of the active substance, e.g., in sesame oil or olive oil.

Following the above treatment and using the described regimen, gynecomastia is inhibited, and in some cases completely prevented.

What is claimed is:

1. Use of an aromatase inhibitor selected from exemestane, anastrozole, letrozole and fadrozole in the preparation of a medicament for use in treating gynecomastia in a patient undergoing a simultaneous, separate or sequential treatment with an antiandrogen.

2. Use according to claim 1, wherein the aromatase inhibitor is exemestane.

3. Use according to claim 1, wherein the antiandrogen is bicalutamide.

4. Use according to claim 1, wherein the patient is undergoing a simultaneous treatment with the antiandrogen.

5. A method of treating gynecomastia in a patient being treated with an antiandrogen, which method comprises administering to a patient in need thereof a therapeutically effective amount of an aromatase inhibitor selected from exemestane, anastrozole, letrozole and fadrozole, or a pharmaceutical composition comprising a said aromatase inhibitor.

6. The method of claim 5 wherein the patient is suffering from prostatic cancer or benign prostatic hypertrophy.

7. The method of claim 5 wherein the aromatase inhibitor is exemestane.

8. The method of claim 5 wherein the antiandrogen is selected from bicalutamide, cyproterone acetate, flutamide and nilutamide.

9. The method of claim 5 wherein the antiandrogen is bicalutamide.

10. The method of claim 5 wherein the antiandrogen and the aromatase inhibitor are administered simultaneously.

11. The method of claim 5 wherein the antiandrogen bicalutamide is administered in a daily dosage of 50 mg and the aromatase inhibitor exemestane is administered in a daily dosage of 25 mg.

12. A kit comprising, in suitable container means, a pharmaceutical composition containing, as an active agent, an antiandrogen and a pharmaceutical composition and containing, as an active agent, an aromatase inhibitor selected from exemestane, anastrozole, letrozole and fadrozole, wherein the aromatase inhibitor is for use in treating gynecomastia induced by the antiandrogen.

13. A kit according to claim 12, wherein the aromatase inhibitor and the antiandrogen are present in distinct container means.

14. A kit according to claim 12, wherein the antiandrogen is bicalutamide and the aromatase inhibitor is exemestane.

15. A product containing an antiandrogen and an aromatase inhibitor selected from exemestane, anastrozole, letrozole and fadrozole, for separate simultaneous or sequential use in the treatment of gynecomastia induced by the antiandrogen.
